(19) United States
(12) Patent Application Publication
(10) Pub. No.: US 2009/0222095 A1
(43) Pub. Date: Sep. 3, 2009

(54) Disc implant device

(76) Inventors: Erik Johansonn, Uppsala (SE); Niklas Axen, Jarlasa (SE); Staffan Bowald, Fjardhundra (SE); Sven Olerud, Lanna (SE); Hans Jacobsen, Taby (SE)

Correspondence Address: MINTZ LEVIN COHN FERRIS GLOVSKY & POPEO, ONE FINANCIAL CENTER, BOSTON, MA, 02111, US

(21) Appl. No.: 11/921,331

(22) PCT Filed: May 31, 2006
(86) PCT No.: PCT/SE2006/000643

§ 371 Date: Mar. 12, 2009

(30) Foreign Application Priority Data
Jun. 1, 2005 (SE) 0501254-7

(51) Int. Cl. A61F 2/44

(52) U.S. Cl. 623/17.16

(57) ABSTRACT

A device in a disc implant (2) for positioning between two vertebrae (3,3′) in a spinal column, said disc implant (2) including two contacting elements (14,14′) for contacting each one vertebra and an intermediate articulation device (15) includes at least one sealing element (13,13′) for attachment to a peripheral part of one of the contacting elements for sealing between that element and an adjacent vertebra. The invention also concerns a disc implant.

### FIELD OF THE INVENTION

The invention concerns a device for a disc implant according to the preamble of claim 1 and a disc implant.

### BACKGROUND OF THE INVENTION

For patients diagnosed disc degeneration, surgical operations are performed more and more often for released troubles. The most common operation for these patients today is still fusion, where an ossified connection of the vertebrae is obtained. Also metallic connection devices can be used. The movability then ceases between the vertebrae in question but the patient will become free from pain. As the patient becomes more active and movable, the segments above and below the fused region will, however, be subjected to greater strains. The risk of new symptoms from surrounding segments thereby increases.

As an alternative to fusion, disc implants have been presented. A known disc prosthesis generally consists of two mutually articulated plates that are positioned between two vertebrae instead of the disc. The positioning of a disc implant results in eliminating the disc that causes pain, reinstating the distance between the vertebrae and reinstating movability between them.

In order to obtain sufficient certainty against a disc implant over time moving in an undesired manner from the intended position between two vertebrae, the two mutually articulated plates of a previously known prosthesis are provided with different kinds of projecting engagement means such as fin-shaped elements, pointed elements, pins and like projections for the engagement with the meeting surfaces of the vertebrae.

The operative method that is used for inserting a disc implant requires positioning of the prosthesis from the abdomen side in order to allow access to the vertebral column from the front. The disc to be replaced is cleared out, whereby the vertebrae are drawn apart with the aid of tension pliers.

A disc implant supports great loads, including both intermittent load, shearing and rotating. The contact surface will, however, never be ideal in known prostheses, because of the diverse and individual nature that characterizes the shape and size of vertebrae. I practice for that reason the contact will be in points or in spots between the vertebrae and disc implant. This gives relatively high surface pressure and in particular in the inner, spongy area of the vertebrae that has a limited load capacity. Persons with the diagnosis osteoporosis can therefore not be subject to surgical insertion of disc implants. Also for persons with normal skeleton, today's method is problematic with risk of overstraining, in particular directly after an implant operation.

### Aim and Most Important Features of the Invention

It is an aim of the invention to reduce the limitations of the background art in such a way that the above mentioned problems are reduced.

This is achieved in a device according to the above through the features of the characterizing portion of claim 1.

Hereby it is made possible that a medium is inserted between the implant and the vertebra in such a way that the load is distributed over a larger area of the vertebra. This reduces the risk of local overloading of the vertebra which otherwise, in worst case could lead to a collapsing vertebra. Further, the risk is reduces of movement/sliding between the prosthesis and vertebrae, not least in an early stage during the healing process. After it is insertion, the medium will fill the space comprising the gap between the prosthesis and the vertebra and will subsequently harden. This way is created a load distributing contact surface having the same shape as the surface of the vertebra.

Vertebrae are advanced building elements as concerns strength properties, having a close-grown outer shell of cortical bone, thickness about 1 mm, and a porous, spongy (porous) central region. In order for the vertebra to maintain its strength, both the outer shell and the porous core should be kept intact as much as possible. It is thus not appropriate to chamfer (or in any other way damage) the contact surface of a vertebra against a prosthesis in order to obtain better fit.

Disc implants are manufactured in a plurality of sizes and angles, which gives certain possibility of choice to obtain fit between prosthesis and vertebrae. The variations of the vertebrae with respect to shape and size is, however, great, and further, the contact surface of the vertebra against the disc is characterized by a domed shape which today's disc implants can not be adapted for. Today's prostheses therefore have generally plain surfaces for contacting the vertebrae.

By providing a sealing element for the attachment at a peripheral portion of a contacting elements of the implant for sealing between that element and an adjacent vertebra, is particular is created an enclosed space between the implant and the vertebra inside the sealing element, wherein said medium can be introduced and be retained until it hardens.

As an example of media that come into question can be mentioned bone cement, polymethylacrylate (PMMA), which is well known for use in different surgical operations in the skeletal structure, for example at the insertion of hip joint prostheses. Other suitable per se known media such as like chemical hardening polymer masses and also other compositions can be used within the scope of the invention, such as ceramic cements. Into the cement can also be added different bioactive substances such as for example so called growth factors. Admixture can also be made of agents promoting ingrowth, such as for example hydroxy apatite, which is considered to promote this.

After application and hardening of the medium, in said enclosed space there will be created a plate of the hardened medium, in particular thus of bone cement, which effectively distributes and equalizes the load between the implant and the vertebra such that an essentially more limited surface pressure will act on a large part of the vertebra instead of the harmful, load point by point, where the vertebra has the least strength. In particular this formed plate should extend out to the cortical region of the vertebra for best load distribution.

Injecting the medium/bone cement into the enclosed space can be made through a channel in the vertebra or through a hole in the seal or even in the implant. The medium/bone cement can preferably be applied by being injected to its location with ordinary syringes and needles. According to a variant of a method were the invention is used, a hole for a syringe is hammered in through the vertebra so that it ends in the enclosed space.

Before the application of the medium/bone cement, a space is thus limited between a vertebra and the prosthesis. This room is advantageously separated by means of a strip of flexible, formable material attached to the outer edge/periphery of the prosthesis. Hereby is created an enclosed space between the vertebra and prosthesis that extends as far out against the outer edge of a vertebra and the prosthesis as possible and well runs along the contours in order for medium/bone cement being injected into the space as much as possible to distribute the load also peripherally out to the cortical region of the vertebra having more support capacity.

Possibly the sealing element is provided with an opening for introduction of medium. This also results in that injecting does not have to be made over a hole in vertebra. As an alternative a medium syringe can be pressed through the sealing elements, for example at a thinned part of the sealing element.

By shaping the sealing element with an inward groove for cooperation with the contact element, secure positioning is achieved.

By forming the sealing element with a narrowing lip, which is intended to lie against the vertebra, a flexible contact relative to the vertebra is achieved, whereby, when the lip is directed obliquely outwards, there is created a condition for forming a supporting plate of medium/bone cement having a greater contact area against the vertebra than against the disc implant. In particular a contact portion against the vertebra is formable for appropriate adjustment to the shape of the vertebra. Further, the contact portion of the sealing element should be so stable and formed that it does not fold or deforms during the application.

When the sealing element is integrally formed, so that is cooperates with the two contacting elements of the prosthesis, several advantages are obtained with respect to handling. One advantage is also that the disc implant can be held together into a more easily handled and positionable unit.

The sealing element further ensures that the medium/bone cement does not leave the desired space and for example is pressed out into the articulating part of the prosthesis or flows against the spinal canal, alternatively goes into a blood vessel and other vessels or vessel systems.

Besides, a certain minor pressure can be applied to the medium in the injection step in order to make it capable of adequately filling the distance between prosthesis and vertebra.

The sealing element has preferably a ring shape and is slipped on to the outer edge of the disc implant.

Further advantages are obtained with the other features of the invention.

### BRIEF DESCRIPTION OF DRAWINGS

The invention will now be described in more detail at the background of embodiments and with reference to the drawings, wherein:

FIG. 1 shows a device for positioning of disc implant,

FIG. 2 shows a sealing device according to the invention on a disc implant,

FIGS. 3 and 4a show a sealing element according the invention in perspective view and sectional view respectively.

FIG. 4b shows a variant thereof, and

FIGS. 5a-c show further variants of fastenings for sealing elements.

### DESCRIPTION OF EMBODIMENTS

Like details have been given the same reference numerals.

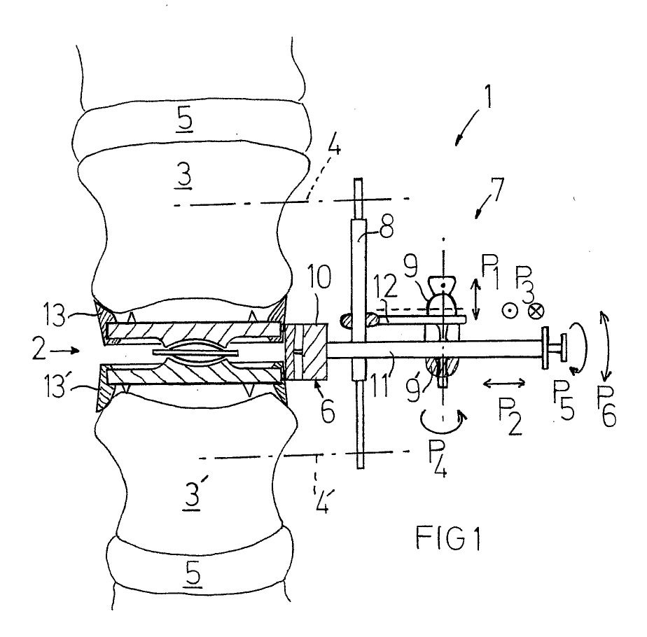

FIG. 1 shows a positioning device 1 in the process of positioning a disc implant 2 between two vertebrae 3 and 3′ in a spinal column of a living human being. With 5 are indicated two healthy discs, whereas between the vertebrae 3 and 3′ is cleared out all material from a damaged disc to be replaced by said disc implant 2.

The disc implant 2 is held by a holding device 6 including a fork-shaped head 10, which releasably grips around the disc implant 2 and a rod shaped manipulating element 11 which can be manipulated by hand of a surgeon.

The manipulating element 11 is controlled by a fixing means 7, which in turn is connected to the distance device 8 (only one shown on FIG. 1), which in turn over engagement means in the form of screws, (indicated with dash dotted lines, and with numerals 4 and 4′), is in engagement with two vertebrae 3, 3′.

The fixing means 7 includes fixing elements 12 (only one shown in FIG. 1) together with a universal joint 9′ which is lockable by means of a locking means 10, wherein the universal joint 9′ in a first, free state allows an adjustment movement including rotations and displacements of the holding means 6 and thereby for the disc implant 2. In a second, locked position, the locking means 9 locks the universal joint 9′ and thereby the holding device 6 and thereby the disc implant 2 in a chosen position.

As is indicated by arrows P₁-P₆, essentially total freedom of movement is achieved with the shown embodiment with three linear degrees of freedom P₁-P₃ and three rotational degrees of freedom P₄-P₆ for the holding device 6. It should be noted that freedom of movement in the length direction of the distance device 8 is obtained by displacement of the fixing element 12 relative thereto. Locking of the locking means 9 can suitably be arranged by means of a smaller rotation of the fixing element 12 with respect of the distance device 8 and thereby friction locking of these elements with respect of each other.

The function of the spanning device of the arrangement is such that the distance device 8 is extendable in the length direction by displacement in such a way that the engagement elements 4, 4′, which thus have been brought to engagement with two vertebrae, because of the extension will cause a change of the degree of separation between these vertebrae 3 and 3′ with respect to each other.

This way the vertebrae can be separated and the space between them be cleared out so that the disc implant 2 without resistance can be inserted between the vertebrae 3 and 3′, accurately be positioned by the surgeon supported by simultaneous X-ray radioscopy until an optimal positioning of the disc implant 2 has been reached. Thereafter the holding device 6 is locked and thereby the prosthesis device in the chosen position with the aid of the fixing means 7, whereafter the distance device 8 is manipulated in such a way that the distance between its outer ends reduces and thereby the vertebrae 3 and 3′ come closer to each other until they come into contact against the outer plates of the disc implant 2. Thereafter a final control is made, by means of X-ray radioscopy or the like, that the disc implant 2 is indeed accurately positioned.

If that should not be the case, the degree of separation is again be increased between the vertebrae 3 and 3′ and the disc implant 2 is repositioned. When accurate positioning has been reached, the holding device 6 is removed from the disc implant 2, whereafter the distance device 8 and its engagement means 4 and 4′ can be removed from the engagement with the vertebrae.

According to the invention, the sealing elements 13, 13′ are arranged on the disc implant in order to seal between that element and the respective vertebra.

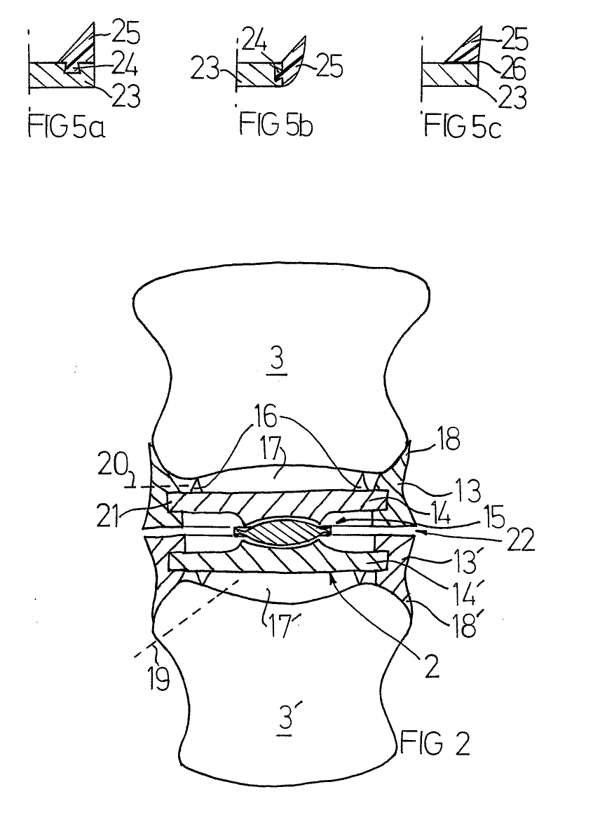

This is shown in more detail in FIG. 2, wherein between two vertebrae 3, 3′ is inserted a disc implant 2 including two essentially plate-shaped contacting elements 14, 14′ with an intermediate articulation device 15. Pins 16 are formed in order to engage into the vertebrae and ensure that the implant 2 remains in its position. Peripherally on the contacting elements 14, 14′ are positioned sealing elements 13 and 13′, respectively.

The sealing elements 13, 13′ have obliquely outwardly directed narrowing lips 18 and 18′, respectively, for contacting the respective vertebra and comprising contacting portions of the sealing elements. In the shown example, the lips contact the outer parts of the meeting portions of the vertebrae, which is preferred, since it gives good load distribution against the cortical portions of the vertebrae, but other variants can come into question, for example if the implant is relatively smaller with respect of the vertebrae than what is the case in FIG. 1.

With 17 and 17′ are indicated spaces that exist between the contacting elements and the vertebrae, outwardly delimited by the sealing elements. The shape of these spaces is very varying depending on vertebra size, position in spinal column etc. The invention makes it possible that a hardening medium such as bone cement is inserted into these spaces and remains there until it has hardened. Hereby is created an adapted contacting between each vertebra and the implant, which results in the great advantages that are mentioned above.

Insertion can be made by the medium being pressed-in by means of a syringe (not shown) through a hole made in the vertebra, which is indicated with an interrupted line at 19. As an example, the syringe needle can be hammered-in through the vertebra so that it debouches in the space 17. As an alternative, medium can be introduced through the sealing itself, which is indicated with interrupted line at 20. Hereby the sealing can also have a hole for this purpose or a simply penetrable inner portion.

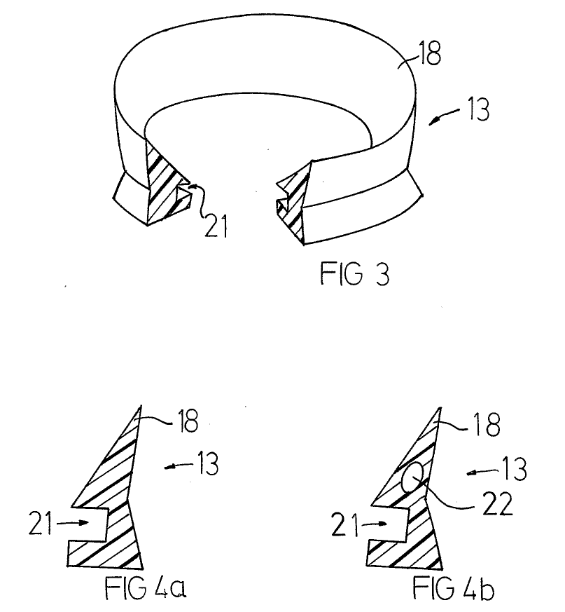

The region at 22 in FIG. 2 is shown free, but it can also be the case that the sealing elements 13 and 13′ are continuous, for example over bridges into one unit as long as it is does not obstruct the articulation function of the implant. FIGS. 3 and 4a show the ring-shaped sealing element 13 in perspective view and cross section view respectively. In the variant in FIG. 4b, the sealing element 13 is provided with a ring shaped chamber 22 in the area of the lip 18 around the entire ring length of the sealing element 13, which chamber is connectable to a device for pressure increase of a fluid being present in the chamber 22. The purpose of this is to increase the volume of the lip 18, increase the contacting pressure of the lip of the sealing element 18 against the vertebra in order to enhance the sealing effect and to fill up possible cavities or the like, altogether in order to ensure better contact of the lip against the vertebra. The chamber can be shaped otherwise, for example have other cross section.

In a method where the invention is used a space between a disc implant and a vertebra is delimited by means of a sealing element vis-à-vis the surroundings. Thereafter bone cement or the like is introduced into this space and is allowed to harden.

The sealing element can be allowed to remain in place inside the patient or be removed by for example cutting-off and pulling away. This depends on chosen material etc. Possibly the adjacent vertebrae are positioned or angled after desire before the insertion of the bone cement. This results in that a more limited number of disc implants have to be used, since varying angles can be taken up by differently angled and shaped medium plates after desire, that are formed between the implant and the respective vertebra.

The invention can be modified within the scope of the claims. For example it can be used also with otherwise constructed disc implants and hereby also other means than the used groove can be used for cooperation with the implant. The contacting portion against the vertebra can be formed otherwise, for example with a plurality of lips, extending concentrically to each other. In order to facilitate filling of cement in the space 17, 17′, an air evacuating channel can be arranged through the sealing element 13 or in any other way. As a variant thereof, inside that channel can be arranged a valve of the kind that is air permeable but is liquid tight.

The sealing element can be attached to the respective contacting element in other ways than what has been described above, whereby generally it has been started out from disc implants that are available of the market today. As an example, with reference to FIGS. 5a and b showing partial cross sections of outer edges of contacting elements, it is possible instead to provide contacting elements 23 with grooves 24, wherein strip portions of the sealing elements 25 protrude for the purpose of fastening. The grooves can for example be placed on the contacting side (axially directed) or on an outwardly directed surface (radically directed). This would result in a more easily manipulated and insensitive solution with pre-fitted sealing elements on the implant.

It is also possible, with reference to FIG. 5c, to use glue joining, heat adhesion etc (indicated with 26) for fixing of a sealing element 25 to a contacting element 23.

The sealing element is suitably from a material from the group: elastomer, elastically formable polymer, natural rubber, synthetic rubber, silicon rubber, silicon oil, natural latex, synthetic latex. It is not excluded that other materials come to use. It is also possible to manufacture sealing elements of resorbable material such as resorbable polymers, elastomers etc. Such sealing elements would after a certain period have been dissolved and resorbed by the body.

It is preferred that the sealing elements are elastic and flexible, respectively, but in certain applications also a sealing element in the form of a rigid, stiff strip comes into question.

1-11. (canceled)

12. A disc implant for positioning between two vertebrae in a spinal column, said disc implant comprising:
at least two contacting elements, at least one each for contacting a respective vertebra;
an intermediate articulation device; and
at least one sealing element included on a peripheral part of at least one of the contacting elements for sealing between that element and an adjacent vertebra.

13. The device according to claim 12, wherein the sealing element is flexible.

14. The device according to claim 12, wherein the sealing element is substantially surrounded by the contacting element.

15. The device according to claim 12, wherein the sealing element is ring-shaped.

16. The device according to claim 12, wherein the sealing element includes an opening for the insertion of a hardening medium into the space between the contacting element and the vertebra.

17. The device according to claim 12, wherein the sealing element includes an inward groove along an extension of the sealing element for receiving a peripheral edge of a contacting element.

18. The device according to claim 12, wherein the sealing element includes a lip along an extension of the sealing element.

19. The device according to claim 18, wherein the lip is directed obliquely outwardly as viewed in cross-section.

20. The device according to claim 12, wherein the sealing element is arranged for enclosing the two contacting elements.

21. The device according to claim 12, wherein the sealing element is made from a material from the group: elastomer, elastically formable polymer, natural rubber, synthetic rubber, silicon rubber, silicon oil, natural latex, and synthetic latex.

22. A spinal disc implant comprising:
at least two contacting elements for contacting a respective vertebra in a spinal column; and
an intermediate articulation device.

23. The disc implant according to claim 22, wherein the sealing element is flexible.

24. The disc implant according to claim 22, wherein the sealing element is substantially surrounded by the contacting element.

25. The disc implant according to claim 22, wherein the sealing element is ring-shaped.

26. The disc implant according to claim 22, wherein the sealing element includes an opening for the insertion of hardening medium into the space between the contacting element and the vertebra.

27. The disc implant according to claim 12, wherein the sealing element has an inward groove along an extension for receiving a peripheral edge of a contacting element.

28. The disc implant according to claim 12, wherein the sealing element has a narrowing lip along its extension for lying against the vertebra.

29. The disc implant according to claim 28, wherein the lip is directed obliquely outwardly as seen in a cross section.

30. The disc implant according to claim 22, wherein an integral sealing element is constructed for enclosing two contacting elements in the disc implant for contacting two adjacent vertebrae.

31. The disc implant according to claim 22, wherein the sealing element is made from a material selected from the group consisting of: elastomer, elastically formable polymer, natural rubber, synthetic rubber, silicon rubber, silicon oil, natural latex, and synthetic latex.

32. A method of treating disc degeneration in a mammalian subject comprising:
providing a disc implant comprising at least two contacting elements, at least one of the two contacting elements for contacting a respective vertebra on either side of the degenerated disc, a sealing element and an intermediate articulation device;
removing a degenerated disc from the mammal; and
replacing the degenerated disc with the disc implant.

33. The method according to claim 32, wherein the sealing element is flexible.

34. The method according to claim 32, wherein the sealing element is substantially surrounding by at least one contacting element.

35. The method according to claim 32, wherein the sealing element is ring-shaped.

36. The method according to claim 32, wherein the sealing element includes an opening for the insertion of hardening medium into the space between the contacting element and the vertebra.

37. The method according to claim 32, wherein the sealing element has an inward groove provided along an extension for receiving a peripheral edge of a contacting element.

38. The method according to claim 32, characterized in that the sealing element includes a narrowing lip along an extension for positioning adjacent the vertebra.

39. The method according to claim 38, wherein said lip is directed obliquely outwardly as viewed in a cross-section.

40. The method according to claim 32, wherein the sealing element is constructed for enclosing two contacting elements in a disc implant and for contacting two adjacent vertebrae.

41. The method according to claim 32, characterized in that the sealing element is made from a material selected from the group consisting of: elastomer, elastically formable polymer, natural rubber, synthetic rubber, silicon rubber, silicon oil, natural latex, synthetic latex.

42. The method according to claim 36, further comprising inserting hardening medium into the space provided for hardening medium.

43. The method according to claim 42, wherein the hardening medium is selected from the group consisting of: bone cement, polymethylacrylate (PMMA), and chemical hardening polymer mass.

44. The method according to claim 42, wherein the hardening medium further comprises a bioactive substance.

45. The method according to claim 44, wherein the bioactive substance is a growth factor.
